(19) United States
(12) Patent Application Publication
(10) Pub. No.: US 2004/0121379 A1
(43) Pub. Date: Jun. 24, 2004

(54) Methods and devices for acquisition of genetic material

(76) Inventor: Mark Peter Ohan, Ramsey, NJ (US)

Correspondence Address: MARK P. OHAN, 8 KNOLLWOOD DR, RAMSEY, NJ, 07446, US

(21) Appl. No.: 10/683,374

(22) Filed: Oct. 10, 2003

Related U.S. Application Data
(60) Provisional application No. 60/417,953, filed on Oct. 11, 2002.

(51) Int. Cl. C12Q 1/68; C12M 1/34

(52) U.S. Cl. 435/6; 435/287.2

(57) ABSTRACT

The invention described herein is a method of collecting genetic material by exposing a protein substrate to a source of genetic material for the purpose of obtaining, procuring, and storing genetic material such as RNA or DNA. The protein substrate is preferably comprised of collagen. The protein substrate is optionally capable of being solubilized or dissolved without disruption of the cell integrity, thus preserving the genetic material within a cell or the genetic material itself and allowing for subsequent analysis. Methods of solubilization of the protein substrate include enzymatic degradation or hydrolysis.

### RELATED U.S. APPLICATION DATA

This utility application is claiming priority to Provisional Patent Application No. 60/417,953 filed with the USPTO on Oct. 11, 2002, by Mark P. Ohan, 311 South Third Ave., Highland Park, N.J., 08904.

### BACKGROUND OF THE INVENTION

Genetic material such as DNA or RNA is often collected, transported, and analyzed, especially in forensic applications, genetic counseling, or paternity matters. Genetic material can be found in cells, bodily fluids, hair, bone, etc. For example, body fluids at a crime scene are swabbed and then transported to a laboratory where the genetic material contained within the cells is extracted and analyzed. Alternatively, cells are acquired through swabbing or scraping of tissues from living humans (i.e. acquiring buccal cells from convicted felons). Traditionally cotton swabs or cellulose membranes are used for the acquisition and storage of cells and genetic material.

Many applications of forensic science and medical research involve the procurement of cells or genetic material from living organisms (animal or plant), deceased organisms, or innate objects that may be contaminated with the genetic material. Typically, the procurement requires scraping or swabbing of surfaces such as the inner surfaces of the mouth, various body cavities, carpeting, clothing, etc. Sources of cells or genetic material may include, for example, blood, biological tissues, serum, plasma, semen, feces, urine, saliva, cerebral fluid, and synovial fluid.

Typically, materials such as polyurethane foam, cellulose, or cotton are attached to a swab and used to collect genetic material. In some cases, subsequent analysis of genetic material (e.g., DNA sequencing) requires the removal of the cells from these materials. The preservation of the cell integrity during removal of the cells or genetic material from the material is essential. It is often difficult and time-consuming to remove cells or genetic material from insoluble materials. A degradable substrate that can be solubilized without disrupting the cell membrane or degrading the genetic material would be ideal.

### SUMMARY OF THE INVENTION

The invention described herein comprises a protein substrate that is used to contact and collect genetic material. The protein substrate can be used alone or can be applied to the source of genetic material by application means such as a stick, swab, or tape. In the preferred mode, the protein is attached to the application means and exposed to the source of genetic material for a period of time sufficient to allow transfer of at least a portion of the genetic material to the protein substrate. Preferably, application means is a stick that is adhered to the protein substrate on at least one end of the stick, thus acting as a swab. Preferably, the protein substrate is collagen or gelatin. The collagen or gelatin can be uncrosslinked or crosslinked and can be adhered to the application means by dipping the application means into a solution or dispersion of the protein and then lyophilizing the protein thereon. Alternative manufacturing techniques may include, but is not limited to, simple dipping and air drying, or attachment of the protein to the application means with an adhesive.

The protein substrate is allowed to contact sources of genetic material such as the inner surfaces of a mouth or other body cavities such as the nostrils, vagina or rectum. Other tissues such as skin, bone, hair, finger or toenails may also be contacted. In addition, the protein may be allowed to contact surfaces such as wood, flora, clothing, or cell culture plates, in order to procure cells or genetic material. It may also be used to wipe or blot biological liquids containing genetic material. Genetic material could also be obtained from exposed tissue or body fluids during a surgical operation or as a biopsy. Also, the protein substrate could be used as a sponge to absorb liquids or act as a filter to remove cells or genetic material from a liquid. The protein substrate would be able to be shaped into molded configurations such as a sphere, film, cylinder, or rectangle. In addition to allowing for the collection of genetic material, the protein substrate may also be employed as a matrix for cells to grow on or within, provided the substrate is subsequently placed in a cell culture media under conditions known to those skilled in the art of cell culture.

After cells or genetic material are collected onto the substrate, the substrate can be degraded or solubilized by treatment with biologically active enzymes such as trypsin, pepsin, collagenase or other suitable proteases. Alternatively, acids or bases may be used alone or with proteases to solubilize or degrade the protein substrate, thereby facilitating release of the genetic material. Alternatively, the genetic material can be rinsed or eluted from the protein substrate without degradation by enzymes. Preferably, the solvent used to solubilize the substrate will have minimal deleterious effects on the cell wall or intracellular constituents.

The manufacturing of such a device involves the generation of a solution or dispersion of a proteinaceous material. The proteinaceous liquid is then cast into a mold that has the final shape and size of the desired device. Subsequently, the liquid can be allowed to air-dry into a film-like material or lyophilized into a sponge-like material. Sticks or handles made of wood, plastic, or metal, can be used as an applicator. After drying, the device may be processed in order to enhance physical properties of material. The device can then be packaged and sterilized.

### BRIEF DESCRIPTION OF THE DRAWINGS

FIG. 1 illustrates the preferred mode of the invention as a stick with the protein substrate attached to one end thereof.

FIG. 2 illustrates the protein substrate as a sponge without application means.

FIG. 3 illustrates the substrate as a film without application means.

FIG. 4 illustrates the protein substrate attached to a tape application means.

### DETAILED DESCRIPTION OF THE INVENTION

I. Protein Substrate

The substrate is designed to be in contact with cells or genetic material and would ideally be protein-based collagen. Alternatively, the protein could be gelatin, albumin, lecithin, immunoglobulin, or other proteins that have a useful biologic function that could be exploited for genetic material acquisition purposes. Various forms of collagen (non-human derived, human-derived, or genetically engineered) could be used. Soluble collagen (individual molecules of collagen in solution) or insoluble collagen (polymeric aggregates of collagen in dispersion) can also be used. Alternatively, soluble or crosslinked gelatin may also be used. Both gelatin and collagen are available as medical grade materials and are currently approved by the FDA for many medical and food applications.

Solutions or dispersions of collagen and gelatin can be cast into films (allowing water to be removed under ambient conditions) or lyophilized into porous sponges (subliming ice into gas). Subsequently, these materials can be fortified and made more durable by crosslinking (introduction of covalent bonds). Methods of crosslinking may include exposure to ultraviolet light, thermal treatment under vacuum, or a variety of chemical means such as formaldehyde, or glutaraldehyde. Alternate configurations of the substrate may include powders, liquids, gels or sprays.

The protein substrate may be used in a fashion that employs simple mechanical collection or entrapment of genetic material, particularly if the source of genetic material is already wet and capable of absorbing or adsorbing to the protein substrate. Alternatively, the substrate can be wet with suitable solvents such as water, saline, or cell culture media to facilitate collection of genetic material, particularly if the source of genetic material is too dry for simple mechanical collection thereof.

II. Means for Application of the Protein Substrate to the Genetic Material

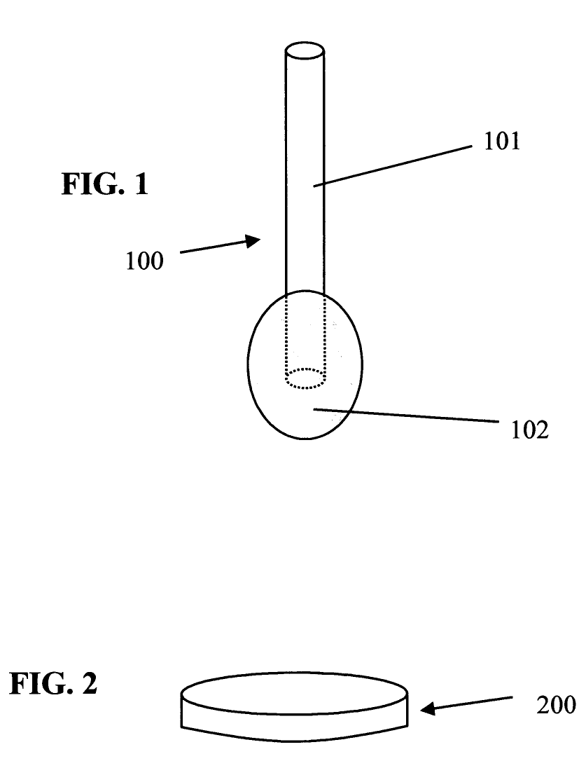

Referring to FIG. 1, the preferred mode is designated by reference number 100. The device 100 involves the substrate 102 directly adhered to a stick or handle 101. The applicator 101 could be made of plastic, wood, protein, or any other material. For example, the stick could be introduced into the solution or dispersion of the substrate and then dried or lyophilized to allow a film or sponge to form directly around the stick. This configuration, resembling a cotton-tipped swab, could be used to swab any surface containing cells or genetic material, for example, the cheek inside the mouth.

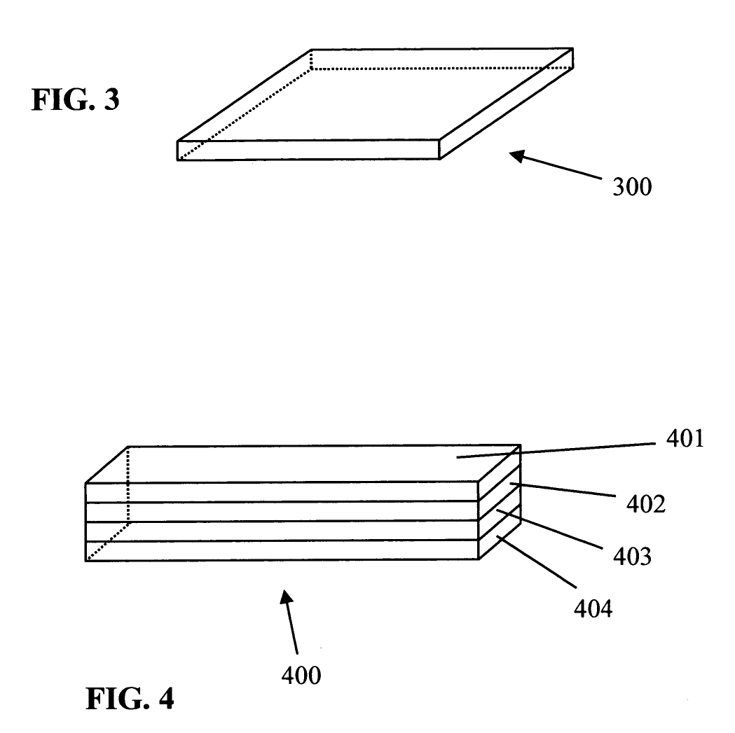

Referring now to FIG. 2 and FIG. 3, the substrate may alternatively be used without an application means. The substrate in the form of a sponge 200 or a film 300 could be used directly to swab, wipe, absorb, etc. any surface containing cells or genetic material. Furthermore, a porous sponge 200 or porous film 300 could be used as a filter to allow for collection of cells or genetic material from liquids containing cells or genetic material.

Finally, referring to FIG. 4, an alternate means of delivery could comprise a multi-layered ‘tape’ 400 wherein the substrate 403 is attached to a backing material 401 by an adhesive 402. A protective layer 404 could be used to cover the substrate 403 to avoid contamination prior to use. When used, the protective layer 404 would be removed; exposing the substrate 403, then the substrate 403 could be placed or rubbed against source of genetic material.

III. Additives to the Protein Substrate

Substances could be added to the substrate during processing or after processing. Adhesive agents could be added directly to the substrate to aid in cell acquisition. Antimicrobial, antiviral, and/or antifungal agents could also be added to prevent contamination of substrate. These agents could include penicillin, triclosan, silver ions, rifampicin, amphotericin, protease inhibitors, etc.

Coloring agents may also be added in order to color code products or to make device more aesthetically presentable. Additionally, flavor agents could be added to those substrates intended for use inside the mouth. Furthermore, plasticizers or hydration agents could be added to substrate (e.g. glycerol, hydrophilic substances, etc.). Also, the device could be supplied either in a dried state or immersed in a liquid (e.g. alcohol, water, acetone, cell culture media, etc.).

IV. Device Packaging and Protection

The device is designed to be supplied as a sterilized, non-toxic material. Packaging materials would be used to protect the device, maintain sterility, and not allow contaminants from coming in contact with device. The packaging would also be used to store the device after the cells or genetic materials have been collected onto the substrate. Examples of packaging and protection materials are glass or plastic ampules or test tubes, and foil pouches or containers made of aluminum, plastic, or Tyvek. After packaging of the device into the packaging, the entire entity can be sterilized with, for example, gamma radiation, ethylene oxide, dry or wet steam (autoclaving), or exposure to electron beams.

V. Solubilization of the Protein Substrate

After cells or genetic material are collected onto substrate, several procedures could follow. In order to liberate the cells or genetic material, a solvent could be used to degrade or solubilize the substrate. The solvent would be directly administered into the packaging containing the device and the acquired cells or genetic material. Examples of solvents include biologically active enzymes (e.g. pepsin, collagenase, pronase, trypsin, etc.), organic solvents, hydrophilic solutions, etc. Other solvents that could be employed are acids, bases, saline, buffers, or Ringers solution. The use of some solvents to solubilize the substrate may involve heating or cooling the solution and device. After the substrate is dissolved, the means of delivery (if any) can be removed and the cells or genetic material can be separated from the solubilized substrate by, for example, centrifugation or filtering. Alternatively, cell culture media could be added to the device and cells in order to initiate cell culturing (e.g. proliferation) of acquired cells that are viable.

### EXAMPLE OF PREFERRED MODE

Fabrication of Substrate on Applicator Stick (Collagen Sponge on a Wooden Stick)

Bovine insoluble collagen (type 1) is dispersed in dilute hydrochloric acid (pH=2.4) at a concentration of 1.0% (weight/volume). An aliquot is poured into a sterile plastic tube (e.g. polypropylene disposable test tube). A handle made of wood is partially immersed into the dispersion and fixed in to place. The device is then frozen at −20° Celsius overnight. Subsequently the device is lyophilized in freeze-dryer. Final product is collagen sponge-like material adhered to the distal end of the wooden stick. The physical properties of the substrate are enhanced by crosslinking with dehydrothermal treatment. This device is then sterilized with a dose of 2.5 Mrads gamma-irradiation.

After cells are collected onto substrate, the substrate is incubated in a buffered saline solution containing trypsin, a biologically active enzyme, at 37° C. until the substrate is solubilized. Subsequently, the cells are collected by centrifugation and then analyzed as desired.

I claim:

1. A method for collecting genetic material comprising; exposing a protein substrate to a source of genetic material; allowing said protein substrate to contact said source of genetic material for a time sufficient to allow for at least a portion of said genetic material within said source to absorb to the substrate.

2. The method of claim 1, further comprising the step of wetting the protein substrate with a liquid selected from the group consisting of water, saline, acid, base, and cell culture media.

3. The method of claim 1, wherein the protein is collagen or gelatin.

4. The method of claim 3, wherein the collagen or gelatin is crosslinked.

5. The method of claim 4, wherein the crosslinked collagen or crosslinked gelatin is in the form of a sponge or film.

6. The method of claim 1, wherein the source of genetic material is tissue from a living human being.

7. The method of claim 6, wherein the tissue is selected from the group consisting of buccal cells, vagina, bowel, nostril, and blood.

8. The method of claim 1, wherein the source of genetic material is selected from the group consisting of blood, urine, semen, hair, skin, bone, sweat, and saliva.

9. The method of claim 1, wherein the substrate further comprises a pharmaceutical agent.

10. The method of claim 9, wherein the pharmaceutical agent is selected from the group consisting of anti-bacterial agents, anti-viral agents, and anti-fungal agents.

11. The method of claim 1 wherein said substrate further comprises flavoring agents or coloring agents.

12. A method for collecting genetic material comprising; exposing a protein substrate to a source of genetic material; wherein said protein substrate is attached to application means, allowing said protein substrate to contact said source of genetic material for a time sufficient to allow for at least a portion of said genetic material within said source to absorb to the substrate.

13. The method of claim 12, further comprising the step of wetting the protein substrate with a liquid selected from the group consisting of water, saline, acid, base, and cell culture media.

14. The method of claim 12, wherein the application means is a stick comprised of a material selected from the group consisting of plastic, wood, and metal.

15. The method of claim 12, wherein the application means is a tape, said tape being comprised of a backing material; and an adhesive layer, said adhesive layer contacting the protein substrate.

16. The method of claim 15, wherein the application means further comprises a protective layer in contact with at least a portion of the protein substrate.

17. A method for collecting genetic material comprising; exposing a protein substrate to a source of genetic material; allowing said protein substrate to contact said source of genetic material for a time sufficient to allow for at least a portion of said genetic material within said source to absorb to said protein substrate; and removing said genetic material from said protein substrate for analysis of said genetic material.

18. The method of claim 17, further comprising the step of wetting said protein substrate with a liquid selected from the group consisting of water, saline, acid, base, and cell culture media.

19. The method of claim 17, wherein said removing step comprises exposing said protein substrate to a chemical selected from the group consisting of acids, bases, water, saline, and protease.

20. The method of claim 19, wherein said protease is selected from the group consisting of trypsin, pepsin, and collagenase.
